(19) United States
(12) Patent Application Publication
(10) Pub. No.: US 2006/0115539 A1
(43) Pub. Date: Jun. 1, 2006

(54) Micropellets method for the production thereof, and use thereof

(76) Inventor: Armin Prasch, (US)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/559,882

(22) PCT Filed: Jun. 3, 2004
(86) PCT No.: PCT/EP04/05993

§ 371 Date: Dec. 7, 2005

(30) Foreign Application Priority Data
Jun. 7, 2003 (DE) 103 25 989.9

(51) Int. Cl. A61K 31/7048; A61K 9/28; A61K 9/50; B01J 13/00

(52) U.S. Cl. 424/490; 514/28; 427/2.14

(57) ABSTRACT

A method for producing micropellets formed of materials that is not easily water soluble, that are provided in the form of a solid dispersion is provided, as well as micropellets which are obtained according to the method, pharmaceutical formulations which contain the micropellets, and the use of micropellets for the production of such formulations. The methods of the invention are based on the use of micronized active material dispersions which can be produced in a specific manner in a fluidized bed process. The micropellets of the invention, also called micropellet cores in their uncoated stage, include, in particular, pharmaceutically effective agents.

### BACKGROUND

The invention relates to a method or a process for producing micropellets, comprising material, that is not easily water soluble, in the form of a solid dispersion, micropellets produced according to the method, a method for producing dispersions comprising material that is not easily water soluble, with the dispersions being used, in particular, for the production of micropellets, pharmaceutical formulations, which contain the above-mentioned micropellets, and the use of micropellets for the production of coated micropellets and/or such formulations. The micropellets, also called micropellet cores in their uncoated stage, include, in particular, pharmaceutically effective agents.

Pharmaceutical, enterally used embodiments shall be formulated in a suitable manner for the respective application, in order to allow the release of the pharmaceutically active agents at the right time and without any disturbing side effects. For example, orally delivered effective agents should be released such, if possible, that no undesired (e.g. bitter) taste develops in the mouth, which would result in defensive reactions, particularly in children, and thus interfere with “compliance.” On the other hand, the active agents shall be released in the stomach or the intestine as complete as possible and in a quickly resorbed form, if a systemic treatment is to occur.

Thus, there is the need to produce granulates, such as micro-spherules, containing pharmaceutically effective agents, with a coating (micro-capsuling).

In classical processes for micro-capsuling of effective agents which are not easily dissolved, this can only be achieved with great difficulty. This is particularly true for the production of granulates made from several components (effective agents, adjuvants), in particular in the case of hard to dissolve effective agents.

IN W/O/W′-emulsifying processes (W represents an aqueous, O a lipophilic phase) an aqueous effective solution is emulgated with a solution of a polymer in an organic solvent that cannot be mixed with water. This W/O-emulsion is subsequently dispersed in a large volume of a polyvinyl alcohol—containing W′—phase, for example. The non-polar polymer solvent disperses in the aqueous phase and the polymer is precipitated as a coacervate. However, in phase separation technology a phase separator is added to a dispersion or an emulsion of the effective agent (e.g., silicone oil), which causes a polymer coacervation of the solvent on the effective agent. After micro particles have formed by way of W/O/W-emulsion or phase separation they are hardened, filtered, and washed in a conventional manner. These processes stress the formulations mechanically and chemically. In extrusion processes a powder mixture made from the effective agent, polymer, and additional adjuvants is heated and pressed through a nozzle. Here, the cylindrical body is formed, which can be further processed. Due to the necessary high temperatures, the effective agents can interact with the polymer, and disintegration processes can occur. Here, as in the case of wet granulation methods, it is very hard to successfully avoid inhomogeneities in the core of the formed particle containing the effective agent. This is caused primarily by the separating phenomenon of the participating components; however, other phenomena also have a negative influence, up to the formation of aggregations of the components, which hinder the release of the effective agent.

Micronization is particularly advantageous for hard to dissolve (in particular in water) effective agents (in particular pharmaceutical ones) because the specific surface of the particles is greater the smaller the individual dimensions of a single particle. Due to the fact that all exchange processes (material exchange and/or heat transfer) in particles occur directly proportional to the surface of the particle, this also influences the behavior of solubility and thus ultimately the bioavailability as well. In particular, when effective agents are provided in a micronized form, particularly hard to dissolve ones, the solubility can be improved considerably by a material exchange surface (=particle surface) being as large as possible, which proves the advantages of micronized materials.

Using classical methods for the production of solid pharmaceutical formulations, particularly smaller particles of effective agents can only be processed with great difficulties, among other things because of their low bulk density, electrostatic charge, etc. Usually, any micronized material is hard to process if at all, because based on its small grain size there is a strong tendency for the formation of dust, lack of flowability, and the mixture with other solid matter, such as necessary components of the formulation of a pharmaceutical product, is only possible with great difficulty. The bulk density of micronized material usually amounts to <0.2 kg/l, frequently even in the range of 0.1 kg/l. Furthermore, any mixture of a micronized effective agent, hard to dissolve in water, with a solvent (e.g., water) by way of conventional agitation (e.g., blade agitator) leads generally to strong frothing and, based on the big differences in density, to a separation of liquid and solid matter. Therefore, a mixture or dispersion as homogenous as possible cannot be produced in this way. This creates problems, for example, in the product handling (dosing, bottling, and the like), during mixing with additional adjuvants or during coating, in order to ensure taste masking and any pH-dependent, controlled release.

Also, in these classical methods it is difficult to yield granulate cores of an even size and a form as round as possible, allowing for example to provide the best coating with other components, such as e.g., taste masking coatings and/or protection coating (for example coatings resisting gastric juice).

From EP 0 163 836 methods for the production of granulates with a narrow distribution of grain sizes is known, primarily used in agrochemicals, which are made in a fluidized bed process. According to examples, they are made either with the use of pure effective agents or by using solvents, melts, or suspension, which optionally may contain inert fillers, dispersing and/or binding agents, and additional material.

### SUMMARY

The object of the present invention is therefore to provide micropellets, provided with the best form, size, and homogeneity of the matrix, in order to allow the production of coated micropellets, which comprise effective agents that are hard to dissolve in water and which help to avoid the above-mentioned problems and disadvantages and which are provided with additional advantages.

The object is attained in a method and a process for producing micropellets containing one or more hard to dissolve effective agents, in which micronized parts are produced by way of spray granulation in the fluidized bed method from dispersions of micronized particles in the presence of a functional adjuvant for the formation of a solid dispersion of such particles, with said functional adjuvants and the other components for the formation of micropellets being provided in a dissolved or also dispersed form.

This is advantageous in reference to the state of the art mentioned at the outset, among other things, in that on the one hand, it does not require the presence of any inert material made from a granulation core. Additionally, the resulting micropellets are provided with numerous other advantages of a surprising combination, for example, a very homogenous matrix structure, high wear resistance, and little dust formation during their production (so that no dust particles develop with their taste being hard to mask, for example). Furthermore, the method according to the invention has the advantage that a high content of effective agent and/or content of functional adjuvants necessary for the formation of a sold dispersion is possible. Further, practically ideal spherical, ball-shaped micropellets develop, which are particularly suitable for a subsequent coating. Homogenous micropellets develop that have a high density and a respectively low porosity. The micropellets are very wear resistant/torn particles are immediately refastened at the core of the micropellet according to the principle of the method. Among other things, the high wear resistance and the low dust formation resulting therefrom allow a very narrow size distribution of the particles without any additional sieving of the micropellets after the pelleting (e.g., from 200 to 400 μM), which again results in a particularly good suitability for any subsequent coating. For example, it is easily possible to achieve that at the most 25% by weight of the micropellets have a diameter deviating by more than 25% (+/−) from the mean diameter of all micropellets. The final product is free from dust, because it is not externally sifted, which again provides the ideal condition for any subsequent coating. The overall yield is very high, for example measured in the distribution of the particle sizes 85 or more %, for example more than 95%.

The primary advantage is the fact that the micropellets are provided with a maximum homogeneity and an ideal suitability for applying (even several) coatings. The solid dispersion of one or more effective agents is a particularly important feature of the micropellets produced according to the method of the invention and results in a considerably increased bioavailability.

Therefore, this surprisingly simple method results in completely novel product features. Even the processing of micronized particles of effective agents, which usually is particularly difficult to process as mentioned at the outset for smaller particles of effective agents, is easily possible.

The miropellets themselves produced by way of the mentioned process form another object of the invention, they comprise one or more hard to dissolve effective agents in a micronized form and one or more functional adjuvants for the formation of a solid dispersion of such effective agents.

The micropellets are particularly suitable for the preparation of pharmaceutical formulations to be administered enterally, in particular orally, on the one hand by processing into tablets, for example coated pills, or dry capsules, on the other hand after coating in form of aqueous suspensions or their preliminary stages in a dry form, which can be suspended by adding aqueous solutions or water so that such formulations are objects of the invention as well.

### BRIEF DESCRIPTION OF THE DRAWINGS

FIG. 1 shows schematically an example of devices used for the production of a dispersion of a micronized effective agent that is hard to dissolve in water.

1 Powder feed, here embodied as a powder funnel; 2 jet stream mixer; 3 charge container; 4 powder wetting device; 5, 6, 7, 8, 9, 10, and 11=valves; 5 valve, for example ball valve for introducing the powder into the arrangement; 6=optional control valve, for example embodied as a ball valve, for feeding additional liquid or another solid matter during the dispersing process, preferably easily and quickly opened and closed, even in an intermediate stage; 7=optional valve, in particular a flap valve, for pumping cleaning agents out of the arrangement after the cleaning, preferably connected to a hose or pipe system for the removal of the cleaning liquid, preferably potential states open or closed; 8 optional (for example, flap) valve, that can be closed during cleaning, potential states preferably open or closed; 9 and 10 each optional service valves, to be closed when necessary in order to prevent that any product already located in the container 3, in the case of service work at the powder wetting device, has to be removed first, preferably embodied as a flap valve and preferably having the settings open or closed; 11 optional valve, for example a flap valve, for the removal of remnants from the pump head and the system subsequent to the decanting phase, its setting preferably either open or closed.

### DETAILED DESCRIPTION OF THE PREFERRED EMBODIMENT

Detailed and preferred embodiments of the invention result in particular from the examples and in general from the claims, the latter being included here by way of reference, and the subsequent explanations:

Preferably, in the micropellets according to the invention one or more pharmacologically effective agents are provided in a micronized form in a portion of 10 to 99% by weight, preferably from 20 to 90% by weight; functional adjuvants for the formation of the solid dispersion in a portion from 1 to 90% by weight, preferably from 1 to 50% by weight, and a desired binder at a portion from 0 to 20% by weight, for example from 5 to 15% by weight, with the sum of these components resulting in 100% by weight.

Effective agents which are hard or not at all soluble in water are suitable, for example, without limitations for one or more effective agents mentioned in the Red List 2003 for drugs, Editio Cantor Verlag, Aulendorf 2003 (incorporated herein by reference as if set forth), and/or in particular one or more of the following effective agents that are hard to dissolve in the solvent used (particularly in an aqueous solution);
antibiotics, in particular macrolide antibiotics, such as clarithromycine, erythromycin, azithromycine, roxithromycine, spiramycine, or josamycine, further ketolides, such as telithromycine, further
hard to dissolve in water antiviral therapeutics, e.g., as antiretroviral proteasis inhibitors, such as indinavir, saquinavir, ritonavir, or nelfinavir;
analgetics, such as paracetamol;
cardio-vascular drugs, e.g., as α-antagonists, such as nifedipin;
antiphlogistics, such as glucocorticoides, e.g., cortison, prednisolon or prednisolon acetate;
cancer therapeutics, such as mitosis inhibitors, for example inhibitors of microtubuli-desaggregation such as taxane, e.g., pacitaxel or docetaxel or the like.

Functional adjuvants for the formation of solid dispersions of the effective agents mentioned are preferably solutizers, in particular polyoxypropylene polyoxyethylene condensates or block polymerisates, such as poloxamer, e.g. Pluronic® (trademark of BASF), fatty acid polyglycol ether, such as the solutizers K2® (General Mills, USA), alkylphenol polyethylene glycol ether, such as the solutizer S-12 (givaudan), triglycerides, such as “labrafil M 2375®” (polyoxyethylene glycerin trioleate of the company Gattefossé, Paris), “Miglyol 812®” (triglycerides of saturated fatty acids of the chain length C₈ through C₁₂ of the company Hüls AG, Germany), or tensides, such as anionic tensides, which usually have long chained fatty acids as a hydrophobic component, such as particularly long-chained (primarily C₈-C₁₈)-alcohols, e.g., alkali metal C₈-C₁₈-alkanoyl sulfates, such as particularly sodium dodecyl sulfate or sodium tridecyl sulfate; the sulfates or sulfonates of monoglycerides of fatty acids, such as alkali metal (particularly sodium-) glyceryl sulfate or sodium coconut oil monoglyceride sulfonate; the sulfonates of succinic acid esters, such as sodium dioctyl sulfosuccinate; the alkylsulfoacetates, such as sodium lauroyl sulfoacetate or sodium coconut oil sulfoacetate; salts of sulfoacetic acid, modified by amino ethyl-long chained-fatty acid esters, such as sodium sulfocolaurate; the amides of higher-level fatty acids with short-chained aliphatic amino acids, such as sodium lauroyl sarcosinate or sodium methyl lauroyltauride; and soaps such as sodium, potassium, or triethanol amine salts of fatty acids, for example of lauric acid, myristicic acid, palmitic acid, stearic acid or mixtures therefrom, or cocoa nut oil fatty acids or tallow fatty acids; cationic tensides, which in addition to hydrophobic, aliphatic, aromatic, or alkyl—moieties have a positively charged hydrophilic group (usually quaternary ammonia), e.g., (additionally anti-bacterially effective) tensides benzyl-dimethyl stearyl ammonia chloride or cetylpyridinic chloride; amphoteric detergents, such as mono or dicarboxylized imidazolines of fatty acids, such as sodium lauryl dicarboxy imidazoline or sodium coconut oil dicarboxy imidazoline or triazaelcosan carboxylic acid; or non-ionic tensides, such as ethoxylized sugar ester of higher-level fatty acids, such as polyoxyethylene sorbitane monolaurate, palmitate, stearate, tristearate, monooleate or trioleate, or alternatively or additionally other solutizers, such as e.g. additional ones named in Fiedler, “Lexikon der Hilfsstoffe für die Pharmazie, Kosmetik, und angrenzende Gebiete”, Editio Cantor Verlag, 5^{th} Edition, Aulendorf 2002, page 1060-1061, which are incorporated herein by reference as if set forth; or (less preferred) mixtures of two or more of them to the extent they can be mixed (for example anionic tensides cannot be mixed easily with cationic tensides.)

Binders are particularly in granulates common natural or synthetic binders, (“glue”), e.g., hydroxyalkyl cellulose, such as hydroxy methyl cellulose and hydroxy ethyl cellulose; methyl cellulose; plant gum such as traganth gum, gum arabicum, carayagum, guar gum, xanthan gum, and irish moss; polyvinyl pyrrolidone, polynicyl alcohol, polyvinyl acetate, gelatin, starch, carboxy methyl starch; specially hydrogenated colophony ester; polyurethanes, synthetic polyelectrolytes, such as alkali salt of the polyacrylic acid; polyethylene glycols with a molar weight of approximately 900 or more, e.g., carbowax® 800, 1000, 1450, 3350, 4600, or 8000, inorganic thickening agents, for example inorganic amorphous silicon dioxide, such as hydrogels (e.g., sylodent® 15 or sylodent® 2 by W. R. Grace and Co.), pyrogenic, sublimated or suspended particles of silicon dioxide (such as Aerosil® 200 by Degussa or Cabosil® by Cabot), colloidal magnesium aluminum silicate, dispersed silicon oxide, colloidal silicon oxide or mixture of two or more of said binders, preferably only one of these binders.

Micronized means that the effective agent or agents is or are used in a strongly milled form. Particle sizes below 30 μm are preferred, for example from 0.1 to 30 μm. The micronization of the effective agent occurs for example by way of milling with suitable mills. Particularly suitable are for example air swept mills (previously pre-milled powdery mill feed is injected together with a gas (air, perhaps inert gas to prevent dust explosions, such as nitrogen or argon) under an increased pressure (for example 10 bar) tangentially into the circular milling chamber; by the expanding gas, the powder particles are strongly accelerated (for example 800 m/s) and more or less rotate in the milling chamber by centrifugal force, depending on their mass; by suitable friction and impingements (flow milling) the particles are milled even further, until they are so fine (micronized) (10 to 0.1 μm, micro powder), that it is removed from the milling chamber together with the gas in the center and is precipitated in filter bags; the cooling effect occurring by the gas expansion also allows the micronization of thermally instable compounds) or colloidal mills (here, a conical rotor moves at great speeds in a controllable distance from the mill housing (fractions of a millimeter). The mill feed, suspended in water, is added, passes the narrow gap and is milled by the traverse forces between the blades moistened with water and the stator (housing wall). The particle sizes that can be produced are at best case scenario below 0.1 μm.

The method for producing a micropellet is particularly characterized in that a liquid, preferably aqueous dispersion comprises one or more hard to dissolve micronized active agents, further comprising one or more functional adjuvants for the formation of a solid dispersion (preferably in a dissolved form) and desirably one or more binders (preferably in a dissolved form), injected from the bottom into a fluidized bed that is empty at the beginning of the process;

In that, by the spray granulation of the dispersion, initial seeds for the pellet formation are autonomously formed without any provision of inert material;

and the pellets produced are sifted during the process via a classification device, in particular an air separator, primarily a zigzag-separator according to EP 0 332 031 B1 (this patent is incorporated herein by reference as if set forth) and removed from the separator when a predetermined pellet size has been reached.

The liquid, preferably aqueous dispersion is preferably produced as follows, with this production method for the dispersion representing a particularly preferred embodiment of the invention:

In a first separate step, a homogenous suspension of the micronized effective agent is produced in water, by suspending in water the micronized, hard to dissolve, particularly not-water soluble effective agents, several respective effective agents, or a respective effective mixture by way of a device for powder wetting or dispersing, for example Ystral CONTI TDS-2 (Ystral GmbH Maschinenbau und Processtechnik, Ballrechten-Dottingen, Germany) and a mixer for homogenizing and/or deaerating the dispersion, e.g., a jet agitator of the company Ystral or an Ultra-Turrax of the company Jahnke & Kunkel (Staufen, Germany). Here, attention must be paid that the mixture is simultaneously deaerated and homogenized and that the micronized solid particles do not agglomerate, but remain evenly distributed in the dispersion in the micronized size of the original particles. This can be achieved particularly by a large amount of liquid accepting a small amount of effective agent in a, for deaeration, relatively small mixing chamber (for example having a volume ranging from 10 to 500 mL, e.g., approximately 200 ml). Overall, on the one hand, the mechanical energy input into the entire charge is important for the even homogenization, which is better the higher the concentration of the solid matter. On the other hand, a content of solid matter being too high leads to poor processing reliability. After the introduction of the entire amount of the effective agent, for example, a weight ratio in the range of 1:1 (1 part effective agent in 1 part liquid) to 1:3 is preferred in the overall charge, with in a particular embodiment of the invention this ratio is in the range from 1:1.5 to 1:2.5, for example 1:2 to 1:2.2. After the introduction of the effective agent the transfer occurs into a larger container, and preferably further deaeration occurs with a jet mixer, with attention having to be paid that no additional air is enclosed.

In another separate step a solution of the soluble (in particular water soluble) functional adjuvant and other components for the formation of micropellets is produced, as respectively defined in greater detail above and below, in a (particularly aqueous) solvent, until the solution becomes clear. This can occur in a conventional manner, for example by way of a blade agitator or a mixer with a dissolver disk. As soon as a clear solution is provided the functional adjuvant and the other components are homogenously distributed in the solution.

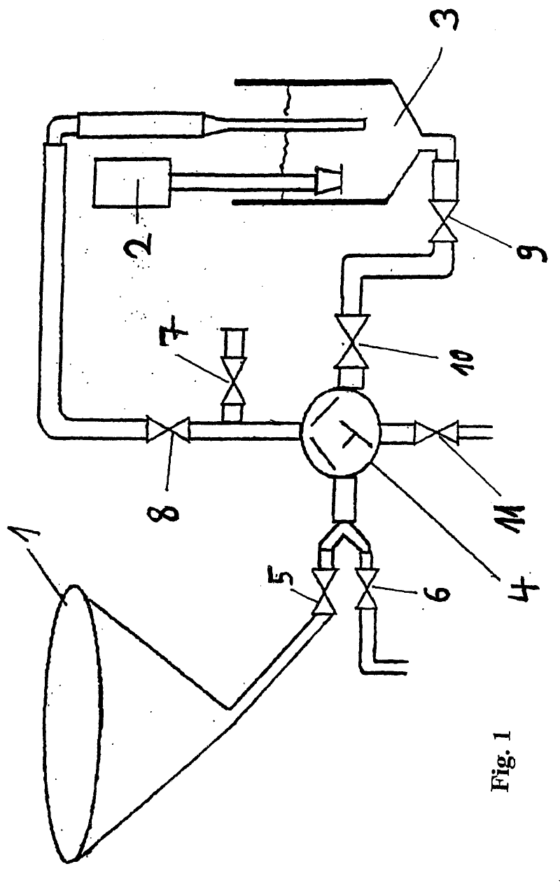

The dispersion of the first step and the homogenous solution of the additional step (if necessary, under the addition of additional solvents, such as water) are subsequently mixed and deaerated in a subsequent step such that a homogenous liquid dispersion develops. This advantageously occurs by way of powder wetting or dispersing devices such, that they suction the homogenous solution in and mix it with the dispersion containing the effective agent, supported by simultaneously mixing and deaerating it with a jet mixer (e.g., from the company Ystral). One example for the device to be used is shown in FIG. 1 (see below in the examples, in which preferred components are described, which can also be used in the process described here in general).

In this manner, distribution of the distributed, micronized particles of the effective agent are distributed in the solution in an ideal manner is achieved, and, thus they are practically surrounded in the solution in an ideal way by functional adjuvants and additional components, such as particularly binders. This condition cannot be achieved by mixing the pure solid matter, in particularly because of the very high air content (in the bulk up to 90% are possible) is to be removed from the micronized effective agent before the contact with the functional adjuvants and the other components occurs, because otherwise a strong, undesired formation of foam would occur.

Preferably, the following process occurs for the production of micropellets: the micronized effective agent, that is hard to dissolve in water or undilutable, or a mixture of two or more such effective agents are suspended in water and subsequently homogenized, so that water and the effective agent are provided in a practically ideal, homogenously distributed dispersion, with this preferably occurring in the manner described above as the “first separate step”, in particular when the solid matter is provided in a powder funnel, suctioning the solid matter into the solvent provided, e.g., water, for example by way of a CONTI TDS-2 of the company Ystral at a speed of 4000 to 6000 rpm's until the entire amount of powder has been suctioned in and homogenization occurs under simultaneous deaeration, for example using CONTI TDS 2 and a jet mixer for the previously determined and set time, e.g., 1 to 60 mins., preferably 10+/−2 mins. The dispersion produced is mixed with a solution (particularly produced in the way described under “separate additional step”) of the soluble (particularly water-soluble) functional adjuvant and additional components and/or additional water (the latter added to the solution earlier, later, or simultaneously, for example) for the formation of micropellet, which are particularly defined in greater detail above and below, (preferably the effective agent, adjuvant, and if necessary, binders are provided in the preferred amounts described above), particularly as described above, in a potentially preferred embodiment, for example by way of the above mentioned CONTI TDS-2. The dispersion developing here (solid matter concentration in such a range that the dispersion can still be pumped or even nebulized, for example from 5 to 40% by weight, for example between 15 and 25% by weight, in particular) is nebulized, and processed into micropellets (preferably after another deaeration subsequent to its addition) in a fluidized bed evaporator (for example in a process and through use of a device according to EP 0 163 836, its content being incorporated by reference herein with respect to the methods and devices used) preferably by using a dual jet. The solvent (water) is removed during the drying process by way of evaporation).

For example, the ratio of effective agent to solvent agent ranges from 20:1 to 1:1, for example in a potentially preferred embodiment from 10:1 to 3:1, e.g., at approximately 4:1. This allows production of micropellets with a high relative content of effective agent.

Micropellets develop (without the addition of core-forming substances as seeds) with a homogenous distribution, comparable to a “solid dispersion”, i.e. the content in effective agent is not present distributed molecularly, rather the distribution is based on the micronized particles. The distribution of effective agent and functional adjuvants for the forming of solid dispersions and, if necessary, one or more binders is equivalent to a homogenous distribution of the liquid dispersion (=matrix system), this way separation phenomena can be avoided effectively.

As soon as such a “solid dispersion” is again brought into contact with a solvent (water, liquids of the gastrointestinal tract lumen), the micropellet immediately disintegrates into the individual micronized solid matter particles of the micronized effective agent. Each individual micronized solid effective agent particle is surrounded homogenously by a functional adjuvant for the formation of the solid dispersion and this way it can be dispersed and even dissolved very quickly. This way, the effective agent can be optimally resorbed and thus the bioavailability is deciding and considerably increased.

The features, in particular the releasing profile of the effective agent, of the uncoated core of the micropellets can be adjusted within the scope of fluidized bed processes without any expensive experimental difficulties by selecting the components and parameters, for example by varying the components of the composition of the core and the adjustment of the size of the micropellets, with the ratio suitable for each respectively desired goal easily being determined by one trained in the art.

The term “releasing profile” relates to the pattern of releasing the respective effective agent over time, for example in the intestines. This can be determined either in vivo, as a measure for the bioavailability, for example by determining the blood count of the effective agent, or preferably ex vivo, for example by way of the “USP paddle”—method, which allows a determination of the dissolution rate of the effective agent.

By a suitable variation of the parameters (composition, fluidized bed method) suitable micropellets can be produced particularly for further processing (e.g., sieving, mixing, dosing, and coating). In contrast to the extrusion method, spherical particles of a small size are achieved with a concentric, homogenous structure of the matrix. Even a single-vessel mixer with subsequent evaporation or even a common fluidized bed granulation cannot achieve similar results.

For example, a USP paddle-device is used at 37° C. and 30 to 100 rpm (revolutions per minute), for example at 75 rpm, and the micropellets according to the invention (uncoated or coated) are examined in (for example 900 ml) artificial gastrointestinal liquid, e.g., phosphate buffers at a pH of 6.6, artificial gastric juice, such as 0.1 N HCl, or water. At predetermined times (e.g., 1, 2, 5, 10, 15, 20, 25, 30, and 60 minutes) samples are taken and the amount of effective agent released is determined by way of standard methods, such as HPLC or spectrophotometry.

For example, in a potentially preferred embodiment of the invention, the particles disintegrate at 37° C. under the above-mentioned conditions so that after 15 mins. 75% or more of the effective agent is released (in a dissolved and/or micronized form), after 30 mins. 85% or more, and after 45 mins. 95% or more.

Preferably, the micropellets produced according to the invention have a particle diameter of less than 600 μm, for example between 10 and 550 μm, for example between 200 and 400 μm.

The micropellets according to the invention can directly be processed, or directly processed and coated to form pharmaceutical preparations.

The micropellets can be filled directly into dry capsules or they can be processed into tablets, in particular coated pills, together with other adjuvants. For dry capsules, for example, hard capsules made from gelatin are used, or soft, sealed capsules made from gelatin and a softener, such as glycerin or sorbite. Other adjuvants can be added to the micropellets, for example fillers, such as corn starch, binders, or lubricants, such as talcum or magnesium stearate, and, if desired, stabilizers such as preservatives.

As adjuvants for tablets, conventional adjuvants are used, for example carrier substances, such as fillers, e.g., sugar, such as lactose, saccharose, mannitol, or sorbitol, cellulose preparations and/or calcium phosphate, such as tricalcium phosphate or calcium hydrogen phosphate, and also binders such as starch, e.g., corn, wheat, rice, or potato starch, methyl cellulose, hydroxy methyl cellulose, sodium carboxy-methyl cellulose and/or polyvinyl pyrrolidone; and if desired, explosives, such as the above-mentioned starches, also carboxy methyl starch, cross-linked polyvinyl pyrrolidon, algine acid, or a salt therefrom, e.g., sodium alginate. Additional adjuvants are in particular flow regulators and lubricants, e.g., silica acid, talcum, stearic acid or salts therefrom, such as magnesium or calcium stearate, and/or polyethylene glycol or derivatives therefrom.

The cores of the tables can be provided with suitable, if desired, gastric acid resistant coatings, for example using concentrated sugar solutions, comprising gum arabicum, talcum, polyvinyl pyrrolidone, polyehtylene glycol, and/or titanium dioxide, or lacquers in suitable organic solvents or solvent mixtures, or for the production of gastric juice resistant coatings, solutions for suitable cellulose preparations, such as acetyl cellulose phthalate or hydroxy propyl methyl cellylose phthalate. Colors or pigments can be added to the tablets or the tablet coatings, for example for identification purposes and in order to indicate different dosages of the effective agent.

On the other hand, the micropellets described are particularly suitable, as already mentioned, for the applying taste masking and/or gastric juice resistant coatings. The coating is preferably in a single or multiple (for example double) layer.

Preferably, directly on the core of the micropellets (with the effective agent) a protective coating is located in order to ensure the separation of the core of the micropellet containing the effective agent from another exterior gastric juice resistant, taste masking exterior coating (because when directly applying a gastric juice resistant coating, a partial “solution” of the effective agent can occur and thus a partial diffusion of the effective agent at the surface of the coated micropellets, which results that in very bitter tasting effective agents a secure taste masking can no longer be achieved).

As a protective coating on the core, for example a coating with a film former (applied in an aqueous or organic solution) can be provided, e.g., cellulose derivatives, such as hydroxy ethyl cellulose, hydroxy propyl methyl cellulose, cellulose acetate dibutyl or cellulose acetate dicyclohexyl aminohydroxy propyl ether or cellulose acetate phtalate, acrylate or methacrylate polymers, mixed polymers made from alkyl, such as butyl methacrylate and dimethyl aminomethacrylate, shellack, polyvinyl pyrrolidone, prolamine, polyvinyl acetate, methacrylic acid morpholine-N-B-ethylacrylate or acrylic acid morhpholino-N-B-ethylmethacrylate styrolacrylate copolymer, mixed polymerisate of 2-hydroxy ethyl-, 2-hydroxy propyl-, 2-hydroxy butyl, or 4-hydroxy butyl ester of the acrylic or methacrylic acid, poly(vinyl)acetate dialkylamino acetate, or mixtures of saccharose and montomorrillionite or mixtures therefrom. For this purpose, fillers, such as titanium dioxide, silicates, talcum, chalk, urea derivatives, starch, alginates, grain flour or the like can be provided and, if desired, a softener, for example polyethyelene glycol, such as PEG 6000.

Preferably, the coating material or the mixture of coating materials in the protective coating is provided at a ratio (in reference to the total amount of the protective coating) from 30 to 90% by weight, a filler at a ratio from 0 to 40, preferably from 10 to 30% by weight, a softener at a portion from 0 to 30, preferably 5 to 12% by weight.

For example, a lipophilic, particularly gastric juice resistant coating is selected as the exterior coating (which can also be present alone, i.e. without the above-mentioned interior coating), allowing sufficient taste masking and simultaneously a very fast release of the effective agent in higher pH-values, in particular at pH 6.8 or higher, with the composition, in particular being characterized in a combination of a lipophilic separator with a surface-active substance as a solutizer in the presence of a film forming component.

In particular, one of the above-mentioned coating agents is used as the film forming component in the exterior coating to the extent it is resistant to gastric juice, or preferably an alkyl acrylate polymer, such as eudragit L 30 D-55® (Röhm) (copolymerisate made from methacrylic acid and ethacrylate at a rate of 1:1).

For example, an ester, for example a tri-C₁-C₇-alkylcitrate such as diethyl citrate, can be used as the separating agent, for example at a weight ratio from 1:60 to 5:1, or other substances forming homogenous aqueous emulsions, or mixtures of two or more thereof.

The lipophilic separating agent in the exterior coating is preferably provided, in reference to the weight portion of the components of the exterior layer, at a ratio from 0.05 to 50% by weight, the film forming component at a ratio from 40 to 99.05, with these components combined resulting in 100%.

The coating as the subsequent step after the production of the micropellets, as described above, occurs preferably also in the fluidized bed method (described according to the Wurster process, for example in a potentially preferred embodiment of the invention in a fluidized bed device according to U.S. Pat. No. 5,236,503 and U.S. Pat. No. 5,437,889, which are incorporated herein by reference); here the coating liquid is nebulized parallel to the micropellets to be coated by way of nozzles in the floor of the fluidized bed liquid, in which the coating agent is dissolved or emulgated. It is particularly beneficial if the nozzle is embodied such that any contact of small particles at the nozzle is prevented. This is achieved, for example, by a cylindrical pipe open towards the bottom surrounding the nozzle, which causes the processing air accelerated in the pipe forming an air pocket around the nozzles, which prevents particularly small particles to contact the nozzle.

Here, for example, first an (interior) protective coating is applied with the above-mentioned preferred components, subsequently (in the same charge or subsequent to an intermediate isolation of the product=single coated micropellets) one or more additional coatings, preferably one additional exterior coating, preferably as described above. Alternatively, only one of the coatings called exterior coating is applied once or several times.

By the above-mentioned coating methods an even coating of the micropellets is possible. One or even two or more coatings can be applied evenly and completely on the pellet surface as a very thin film. Due to the fact that the micropellets according to the invention, as described above, have very advantageous features for the coating, the amount of coating material can be minimized, which is very advantageous particularly for the coating of small particles/micropellets.

This results in the following advantages of the products:

little use of coating material
short processing time
thin film thickness ultimately result in small pellet sizes for the micropellets described. This is important in connection with the desired small particle sizes, such as required, for example in drink suspensions,
targeted and simple application of multi-layered coatings (e.g., double coating).

A process is preferred which includes both the above-described production of micropellets as well as their coating, comprising in particular two coatings, a protective coating and an exterior layer.

Particularly preferred are coated micropellets produced according to this method. Another preferred embodiment of the invention relates to pharmaceutical formulations, which include uncoated or particularly coated micropellets produced as described according to the invention. Here, pharmaceutical formulations are focused on, that are administered enterally, in particular orally, either in the form of drink suspensions or suspensions inserted via tubing directly into the stomach or intestinal tract or (for rectal application) suspensions for enemas or the like, or in the form of suspensions for orally administered capsules, or for tablets, or for the production of such pharmaceutical formulations. These formulations are produced according to conventional methods.

By selecting the participating components a pH-dependent release as fast as possible at high pH-values can be achieved (such as for example present in the intestines), for example pH-values of 6.8 or higher, while at low pH-values, for example pH 5.5 or lower, no release occurs.

The invention can also relate, in another embodiment, to a device as shown in FIG. 1 and/or as generally described in the description of FIG. 1 above and its use for dispersing micronized effective agents, as described above and below, in particular within the scope of the production of micropellets according to the invention.

The above-mentioned definition of certain terms can also be used individually or in combination for a definition in greater detail of general terms in the claims or other embodiments of the invention, which leads to a particularly preferred embodiment of the invention.

Particularly preferred are the embodiments of the invention mentioned in the examples.

The following examples serve to illustrate the invention without limitations (all % values are given in % by weight):

### EXAMPLE 1

### Micropellets With Macrolide Antibiotics (e.g., Azithromycin or Particularly Clarithromycin)

Using the fluidized bed method, the following micropellets are produced:

Production of a Liquid Dispersion Comprising Effective Agents Hard to Dissolve in Water

References are given in FIG. 1

Micronized macrolide antibiotic (e.g., azithromycin or particularly clarithromycin) (grain size<30 μm) is introduced in form of a powder by way of a CONTI TDS-2 (=powder wetting device of the company Ystral) 4 (see FIG. 1) from a powder funnel into water provided (twice the amount of water, i.e. amount of the effective agent 12 kg, amount of water 24 kg), with attention being paid to the fact that no air is introduced along with it, and subsequently it is mixed with a jet stream mixer (jet stream mixer of the company Ystral) 2, homogenized (duration 10 mins.) and deaerated.

This occurs particularly with the device shown in FIG. 1. For starting the operation, valve 9 is opened, all other valves are closed. A coolant supply system for a lubricant seal (both of them not shown) is switched on. When a pressure gauge (not shown) registers a sufficiently high pressure in the coolant supply pipe the pump is released. Valve 8 is opened and the pump rotation is adjusted between 4000 and 6000 rpm. Simultaneously the jet mixer is switched on at 1500 to 5000 rpm. In order to introduce the effective agent, valve 5 is opened until the effective agent has been suctioned through the powder funnel 1. During the introduction of the powder, an interval tapper (not shown) is activated at the powder funnel. For the homogenization, valve 5 is closed and the CONTI TDS-2 is adjusted between 4000 and 6000 rpm. The temperature of the suspension is monitored in order not to exceed a certain value depending on the viscosity (e.g., 60° C.). Valve 5, embodied as a ball valve, can be opened and closed very quickly (in order to allow a quick interruption of the introduction process, for example in the event of channel formation in the funnel with the risk of introducing air) and is opened for introduction into the arrangement.

In a second charge container, an aqueous tenside solution with an additional binder is provided and dissolved: provided water (61.225 kg) is mixed with the tenside (3 kg poloxamer 188=pluronic®) and 2.045 binder (polyvinyl pyrrolidone).

The clear solution is added via the CONTI-TDS-2 to the dispersion of the effective agent and mixed using the jet stream mixer, homogenized and deaerated. Here, the CONTI TDS-2 is operated at a rotation from 2000 to 6000 rpm. The valve 6 is opened until the desired amount of tenside solution has been introduced with the binder. Subsequently, the valve is closed. Here, the jet stream mixer operates at a rotation from 300 to 1500 rpm.

A repeated run of the following mixing and deaeration sequence follows: the mixing is first performed with the CONTI TDS-2 at 4000 to 6000 rpm. The subsequently effective jet stream mixer operates at a rotation from 3000 to 5000 rpm.

While opening the valves 8, 9, 10 the product is pumped into the charge container 3. For removing any residue from the CONTI TDS-2, valve 11 is opened and a suitable collection vessel is held underneath the outlet. The CONTI TDS-2 and the jet stream mixer are switched off.

Subsequently, the liquid dispersion produced in this manner is directly nebulized for the production of pellets.

2. Production of Pellets

The production of pellets occurs by way of spray granulation, by atomizing the liquid dispersion of the effective agent from the bottom into the empty fluidized bed arrangement. As soon as the particles reach the desired particle size, they are removed from the arrangement by way of a zigzag-separator (see, for example, EP 0 163 836, EP 0 332 031). Preferably, GPCG 30 with a WSA-module is used (fluidized bed—spray agglomeration) (both available from Glatt GmbH, Binzen, Germany) (GPCG—glatt particle coater granulator) for the arrangement.

Draft air temperature: 120° C.

Draft volume: 550 m³/h

Product temperature: 72° C.

The targeted size for the pellets ranges from 200 to 400 μm.

The composition of the uncoated pellets (assuming 100% macrolide antibiotic in the original charge): macrolide antibiotic 70%, pluronic® 18%, polyvinylpyrrolidone K30 12%.

3. Coating (Taste Masking)

Applying a layer of coating occurs in a GPOG 30 with a 18 “HS Wurster (HS=high speed wurster, cf. U.S. Pat. No. 5,236,503 and/or U.S. Pat. No. 5,437,889; company Glatt, Binzen, Germany)

Provided amount of pellets: 25 kg

Coating amount applied: 12.5 kg (equivalent to a weight increase of 50% in reference to the pellets)

Composition of the Coating Material

Eudragit® L 30 D 55 (Degussa Co.) 83.89%

Triethyl citrate (Morflex Co.) 12.58%

Glycerol monostearate (Cognis Co.) 2.52%

Tween 80 (Uniquema Co.) 1.01%

Process Parameters:

Draft temperature 70° C.

Draft amount 1000 m³/h

Product temperature 42° C.

The results of the in-vitro release: According to the above-described US-paddle method, more than 75% of the effective agent is released at 37° C. and 75 rpms. The resulting coated pellets show only a slight bitterness and, thus a tolerable taste masking.

### EXAMPLE 2

### Two-Layer Coating

1. coating: polyvinyl-pyrrolidone organic

2. coating: eudragit L30 D-55+10% trietyl citrate

Micropellets produced according to the process of example 1 are provided according to the Wurster process subsequently with the 1^{st} (interior), then the 2^{nd} (exterior) coating.

Result: improved taste masking in reference to example 1; release profile within the scope of the US-paddle test: more than 75% after 15 mins. at 75 rpm and pH 6.8.

1. A method for the production of micropellets comprising one or more hard to dissolve effective agents, the method comprising producing micronized particles of the effective agents from dispersions with functional adjuvants for the formation of a solid dispersion of the particles by spray granulation in a fluidized bed process, with the functional adjuvants and other components for the formation of the micropellets being provided in a dissolved or dispersed form.

2. A method according to claim 1, wherein a weight ratio of the functional adjuvants for formation of the solid dispersion to the effective agent ranges from 20:1 to 1:100.

3. A method according to claim 1, wherein the effective agent is provided in a micronized form with a grain size of 30 μm or less.

4. A method according to claim 1, wherein one or more solutizers are provided as the functional adjuvants for the formation of the solid dispersion, comprising one or more polyoxypropylene polyoxyethylene condensates, fatty acid polyglycol ether, alkyl phenol polyethylene glycolether, triglycerides, anionic tensides, cationic tensides, amphoteric detergents or non-ionic tensides, or a polyoxypropylene oxyethyelene (block)polymerisate.

5. A method according to claim 1, wherein one or more effective agents are provided as the hard to dissolve effective agents, selected from one or more of macrolide antibiotics, comprising azithromycin, antiviral therapeutics which are hard to dissolve in water, analgetics which are hard to dissolve in water, cardiovascular medications which are hard to dissolve in water, antiphlogistics which are hard to dissolve in water, and cancer therapeutics which are hard to dissolve in water.

6. A method according to claim 5, wherein clarithromycin is provided as the hard to dissolve effective agent.

7. A method according to claim 1, wherein the solid matter to be pelletized is provided as a liquid dispersion, comprising the micronized effective agent and the functional adjuvants for the formation of the solid dispersion and a desired binder, injected from a bottom into a fluidized bed arrangement which is empty at a beginning of the process;
starting seeds for pelletizing being formed by way of spray granulation of the dispersion without the presence of any other inert material; and
the micropellets produced during the process being sifted via a classification device, and being removed from the separator when reaching a predetermined pellet size.

8. A method for the production of a dispersion of a micronized effective agent, wherein
in a first separate step, a homogenous suspension of the micronized effective agent is produced in water, by suspending the micronized, hard to dissolve, not water-soluble effective agent, several respective effective agents or a respective mixture of effective agents using a powder-wetting or dispersing device and by a mixer for homogenizing and/or deaerating the dispersion in water under deaeration and homogenization;
in another separate step, mixing a solution of the soluble functional adjuvants and other components for the formation of micropellets is mixed in a solvent, until the solution becomes clear;
and mixing the dispersion of the first step and the homogenous solution of the other step with one another and deaerating in a subsequent step such that a homogenous liquid dispersion develops, advantageously using powder wetting or dispersing devices, with the homogenous solution being introduced by the device and mixed with the dispersion containing the effective agent and the mixture and the deaeration being simultaneously carried out by a jet stream mixer.

9. A method according to claim 7, wherein the dispersion is nebulized in a fluidized bed evaporator, with the solvent being removed during a drying process through evaporation for the production of micropellets.

10. Micropellets produced according to the method according to claim 1.

11. A method according to claim, 1 comprising the micropellets being produced with the following components:
(i) the pharmacological effective agent in a micronized form at a ratio from 10 through 99% by weight;
(ii) the functional adjuvants for the formation of a solid dispersion at a ratio from 1 through 90% by weight and
(iii) a binder at a ratio from 0 to 20% by weight.

12. A method according to claim 11, wherein the micropellets are produced having a diameter from 0.1 to 500 μm, in spherical form.

13. Micropellets according to claim 11, wherein the micropellets are produced so that no more than 25% by weight of the pellets have a diameter deviating by more than 25% (+/−) from a mean diameter of all of the pellets.

14. A method according to claim 11, wherein the micropellets are produced having a pharmaceutical formulation.

15. A method for producing coated micropellets, comprising the production of a micropellet according to claim 1, wherein after the production of the pellets, a coating is also applied in a fluidized bed process, with nozzles in a base atomizing a coating fluid, in which the coating agents are dissolved or emulgated, in a parallel flow into the micropellets to be coated.

16. A method according to claim 15, wherein after a first internal protective coating, subsequently one or more coatings are applied.

17. Coated micropellets, produced according to the method according to claim 15.

18. Coated micropellets according to claim 16, provided with two coatings, comprising an inner protective coating and an outer coating resistant to gastric juice.

19. Coated micropellets according to claim 17, wherein within 15 minutes the micropellets show a release in effective agent of 75% or more in a US paddle test at 75 rpm in a solution with pH of 6.8 or higher.

20. A method according to claim 15, wherein the coated micropellet comprises a pharmaceutical formulation.
